(19) United States
(12) Patent Application Publication
(10) Pub. No.: US 2008/0025918 A1
(43) Pub. Date: Jan. 31, 2008

(54) Invisible light fluorescent platelets for intraoperative detection of vascular thrombosis

(75) Inventors: John V. Frangioni, Wayland, MA (US); Robert Flaumenhaft, Newton, MA (US)

Correspondence Address: FISH & RICHARDSON PC, P.O. BOX 1022, MINNEAPOLIS, MN, 55440-1022, US

(73) Assignee: Beth Israel Deaconess Medical Center, Inc., Boston, MA (US)

(21) Appl. No.: 11/824,916

(22) Filed: Jul. 3, 2007

Related U.S. Application Data
(60) Provisional application No. 60/818,400, filed on Jul. 3, 2006.

(51) Int. Cl. A61K 49/00; A61P 7/00; C12N 5/06

(52) U.S. Cl. 424/9.6; 435/372; 435/375

(57) ABSTRACT

This invention relates to methods, kits, and compositions for intraoperative detection of aggregates of platelets, e.g., associated with vascular thrombosis, using invisible light fluorophore (IRF)-loaded platelets.

### CLAIM OF PRIORITY

This application claims the benefit of U.S. Provisional Patent Application Ser. No. 60/818,400, filed on Jul. 3, 2006, the entire contents of which are hereby incorporated by reference.

### FEDERALLY SPONSORED RESEARCH OR DEVELOPMENT

This invention was made with Government support under Grant Nos. R01-CA-115296, R21-CA-110185, and R01-HL-63250, awarded by the National Institutes of Health, and a Center for Integration of Medicine and Innovative Technology (CIMIT) Application Development Award awarded by the Department of Defense. The Government has certain rights in the invention.

### TECHNICAL FIELD

This invention relates to methods, kits, and compositions for intraoperative detection of vascular thrombosis using invisible light fluorescent platelets.

### BACKGROUND

Arterial and venous thrombosis is a major complication of surgery. There is an immediate clinical need for a non-invasive method to quantify thrombus location and size intraoperatively and in real-time.

### SUMMARY

The present invention is based, at least in part, on the discovery that invisible light fluorophore (ILF)-loaded platelets are a sensitive reagent for detecting thrombi in the operative setting. Therefore, provided herein are methods of using ILF-loaded platelets for real-time detection of vascular clots during surgery. The methods described herein can also be used, e.g., for detection of vulnerable plaques during angioscopy; distinguishing coagulopathy from surgical bleeding; and identification of retained/accessory spleen during splenectomy. Also included are compositions including the ILF-loaded platelets, and kits for performing a method described herein.

Provided herein are methods that can be used to detect an aggregation of platelets. As one of skill in the art will appreciate, such an aggregation of platelets, depending on its location in the body, may be associated with (i.e., part of) a thrombus, a vulnerable plaque, or a retained or accessory spleen. In addition, an aggregation of platelets is associated with active clotting, and thus can be used to determine whether surgical bleeding is normal (if clotting is occurring, an aggregation of platelets will occur) or due to a clotting disorder (if no clotting is occurring). The presence of a clotting disorder can also be diagnosed using the methods described herein, based on the same principles.

In a first aspect, the invention provides methods for detecting a thrombus, e.g., an intraoperative thrombus, in vivo. The methods include administering to a subject a sufficient amount of a composition comprising invisible light fluorophore (ILF)-loaded platelets; and detecting, e.g., during a surgical procedure, invisible light emission from the ILF-loaded platelets, e.g., from an aggregation of ILF-loaded platelets. The presence of invisible light emission from the ILF-loaded platelets indicates the presence of a thrombus.

In another aspect, the invention provides methods for detecting vulnerable plaques, e.g., plaques that are about to rupture (become thrombotic) in vivo. The methods include administering to a subject suspected of having atherosclerosis a sufficient amount of a composition comprising invisible light fluorophore (ILF)-loaded platelets; and detecting, e.g., using an angioscope, invisible light emission from the ILF-loaded platelets, e.g., from an aggregation of ILF-loaded platelets. The presence of invisible light emission from the ILF-loaded platelets indicates the presence of a vulnerable plaque.

In a further aspect, the invention features methods for detecting retained or accessory spleen in vivo. The methods include administering to a subject a sufficient amount of a composition comprising invisible light fluorophore (ILF)-loaded platelets; and detecting invisible light emission from the ILF-loaded platelets, e.g., from an aggregation of ILF-loaded platelets. The presence of invisible light emission from the ILF-loaded platelets, e.g., in the area of the spleen (e.g., where the spleen is, was, or should be) or in one of the peritoneal folds, indicates the presence of retained or accessory spleen.

In an additional aspect, the invention provides methods for determining whether intraoperative bleeding in a surgical field is due to a clotting disorder or is normal surgical bleeding in vivo. The methods include administering to a subject a sufficient amount of a composition comprising invisible light fluorophore (ILF)-loaded platelets; and detecting invisible light emission from the ILF-loaded platelets in the surgical field, e.g., from an aggregation of ILF-loaded platelets. The presence or absence of invisible light emission from the ILF-loaded platelets in the surgical field indicates whether the bleeding is due to a clotting disorder or is normal surgical bleeding. For example, the presence of invisible light emission from the ILF-loaded platelets in the surgical field indicates ongoing clot formation, which means that the bleeding is normal surgical bleeding. A lack of invisible light emission from the ILF-loaded platelets in the surgical field indicates that there is little or no ongoing clot formation, which means that the bleeding is likely due to a clotting disorder.

Further, the invention provides methods for evaluating a subject for the presence of a clotting disorder. The methods include administering to a subject a sufficient amount of a composition comprising invisible light fluorophore (ILF)-loaded platelets; and detecting invisible light emission from the ILF-loaded platelets, e.g., from an aggregation of ILF-loaded platelets. The presence or absence of invisible light emission from the ILF-loaded platelets indicates whether the subject has a clotting disorder. For example, the presence of invisible light emission from the ILF-loaded platelets indicates that the subject does not have a clotting disorder.

In the methods described herein, the platelets can be autologous to the subject, or allogeneic to the subject, e.g., from an HLA-matched donor. The platelets can be, e.g., fresh, lyophilized, fixed or frozen. In some embodiments, the platelets are in platelet-rich plasma. In some embodiments, the platelets are from a blood-bank or other commercial source.

In some embodiments, the ILF is a near-infrared fluorophore, e.g., IR-786.

In some embodiments, detecting invisible light emission from the ILF-loaded platelets can include illuminating the subject with an excitation wavelength of the ILF; and electronically capturing a IL emission wavelength image of the ILF.

In yet another aspect, the invention provides compositions comprising invisible light fluorophore (ILF)-loaded platelets. In some embodiments, in an ILF-loaded platelet the ILF is concentrated in a membrane, e.g., plasma membrane or an intracellular membrane; in an organelle, e.g., mitochondria, endoplasmic reticulum, or nucleus; in cytosol, or on the surface of the cell. In some embodiments, the ILF is a near-infrared fluorophore, e.g., IR-786 or IRDye78.

The platelets can be, e.g., fresh, lyophilized, fixed or frozen. In some embodiments, the platelets are in platelet-rich plasma. In some embodiments, the platelets are from a blood-bank or other commercial source.

In another aspect, the invention provides methods for preparing invisible light fluorophore (ILF)-loaded platelets. The methods include incubating a composition comprising platelets with an ILF under conditions and for a length of time sufficient for the platelets to take up (i.e., be loaded with) the ILF. In some embodiments, the ILF is a near-infrared fluorophore, e.g., IR-786. The platelets can be, e.g., fresh, lyophilized, fixed or frozen. In some embodiments, the platelets are in platelet-rich plasma. In some embodiments, the platelets are from a blood-bank or other commercial source.

Also provided herein are kits for preparing invisible light fluorophore (ILF)-loaded platelets. The kits include a container including a sterile composition that includes an ILF and instructions for use in a method described herein.

An “Invisible light fluorophore” (ILF) is a compound that emits light at wavelengths above those visible to the human eye, i.e., above 670 nm, e.g., up to 10,000 nm. ILFs fluoresce in the invisible light region of the spectrum (680 nm to 100,000 nm), such as near infrared (670 nm to 1000 nm) to mid infrared (1000 nm to 20,000 nm) to far infrared (20,000 nm to 100,000 nm), as any light above 670 nm is invisible to the naked human eye. These invisible light fluorophores do not substantially change the appearance of the surgical field, and because tissue autofluorescence at these wavelengths is generally low, detection is extremely sensitive. Hence, invisible light fluorophores are ideal reagents for surgical imaging. In some embodiments, ILFs can also include fluorophores that are visible to the naked human eye, as long as they also fluoresce in the invisible light region.

The invention provides several advantages. The methods described herein provide real-time, sensitive detection of thrombi. Autologous platelets can be used, lessening any risk of immune or reaction. In addition, the methods described herein are fast and simple, lessening the risk of false positives or false negatives.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

This patent or application file contains at least one drawing executed in color. Copies of this patent or patent application publication with color drawing(s) will be provided by the Office upon request and payment of the necessary fee.

FIG. 1A is a schematic illustration of the chemical structure of the cationic, lipophilic heptamethine indocyanine IR-786.

FIG. 1B is a line graph illustrating IR-786 incorporation into platelets as a function of time at room temperature (RT), using an extracellular concentration of 2 μM IR-786.

FIG. 1C is a line graph illustrating the relationship between final intracellular concentration of IR-786 (attomole per platelet) and extracellular concentration of IR-786 after incubation for 30 min at RT.

FIGS. 1D and 1E are phase contrast (1D) and NIR fluorescence (1E) photomicrographs of platelets loaded with 2 μM extracellular IR-786 for 30 min at RT, and settled on glass slides.

FIGS. 1F and 1G are aggregometry tracings of washed platelets incubated with either DMSO (vehicle control) or 2 μM IR-786 for 30 min at RT, then stimulated with either 1 U/ml thrombin (1F) or 2 μg/ml collagen-related protein (CRP; 1G). Representative aggregometry tracings are from n=4 independent experiments.

FIG. 2A is a line graph illustrating time course of clearance of IR-786-loaded platelets from the circulation. 3.6×10¹⁰ autologous washed platelets were loaded with 2 μM IR-786 for 30 min at RT and infused back into a Yorkshire pig. Platelets isolated from blood samples taken at the indicated times were quantified for NIR fluorescence. Shown are mean ±SEM for n=3 animals.

FIGS. 2B and 2C are aggregometry tracings illustrating effects on platelet function. Washed pig platelets were incubated with either DMSO (negative control) or 2 μM IR-786 for 30 min at RT (positive control), and compared to washed platelets isolated from the bloodstream during FIG. 2A at 2 hours post-infusion (2 hrs). Platelet samples were stirred in an aggregometer and stimulated with either 5 U/ml thrombin (2B) or 4 μg/ml collagen-related peptide (CRP; 2C). Representative aggregometry tracings are from n=3 independent experiments.

FIGS. 3A-3F are images showing real-time detection and quantification of thrombus formation, 1 hour post-FeCl₃-induced injury of the femoral artery (3A-3C) or femoral vein (3D-3F). Shown are color video (3A, 3D), NIR fluorescence (3B, 3E; 67 msec exposure time), and a pseudo-colored (lime green) merge of the two (3C, 3F). Arrow indicates the location of intravascular thrombus.

FIG. 3G is a trio of images of H&E histology (bottom left) and NIR fluorescence (bottom right) from the same tissue section of a FeCl₃-induced thrombus in the femoral artery shown at top. Note characteristic changes to the vessel wall exposed to FeCl₃.

FIG. 3H is a trio of images of H&E histology (bottom left) and NIR fluorescence (bottom right) from the tissue section of an injured femoral artery shown at top.

FIG. 4A is a panel of twelve photographs illustrating detection of intravascular thrombi following surgical injury or insertion of intravascular devices. Various clinically-relevant sites of thrombus formation were imaged in real-time using color video (left column), NIR fluorescence (middle column; 67 msec exposure time) and a pseudo-colored merge of the two (right column). Shown are skin 5 minutes after scalpel incision (top row), carotid artery with electrocautery (EC) burn-induced thrombus (second row), iliac artery 5 minutes after placement of an embolic coil (third row), and ileac artery 45 minutes after placement of a stent without systemic anti-coagulation (bottom row).

FIG. 4B is a bar graph illustrating the time delay between vascular injury and the first detection of a NIR fluorescent thrombus was measured after FeCl₃ treatment of the femoral artery (FA), FeCl₃ treatment of the femoral vein (FV), and embolic coil placement in the femoral artery. Shown are mean ±SEM for n=6 animals for embolic coil and n=8 animals for FeCl₃. First detection was defined as an intravascular NIR fluorescent signal with an SBR≧1.5 in an area≧1 mm².

FIG. 5A is a line graph illustrating real-time quantification and visualization of thrombolytic therapy. The effect of intravenous streptokinase (125,000 IU) and heparin (10,000 IU) infusion on a thrombus formed by an embolic coil placed in the femoral artery. Shown are the mean ±SEM SBR of the NIR fluorescence signal over the femoral artery for n=3 animals. Representative NIR fluorescence images corresponding to the indicated time points of one animal are shown. NIR fluorescence images have identical exposure times (67 msec) and normalizations.

FIG. 5B is a line graph illustrating real-time quantification and visualization of thrombolytic therapy in a femoral artery treated with FeCl₃ at t=0. Shown is embolization of an intravascular thrombus 12 minutes after intravenous infusion of streptokinase (125,000 IU) and heparin (10,000 IU). Shown are the mean SBR of the NIR fluorescence signal over the femoral artery, along with NIR fluorescence images corresponding to the indicated time points. NIR fluorescence images have identical exposure times (67 msec) and normalizations.

FIG. 6 is a panel of sixteen images illustrating assessment of vascular patency and thrombus formation using dual channel NIR fluorescence imaging. IR-786 loaded platelets fluorescing at 800 nm were used to detect intravascular thrombus (pseudo-colored in lime green; white arrows) and methylene blue fluorescing at 700 nm was used to assess blood flow (pseudo-colored in yellow). The dotted black arrow shows the direction of blood flow. Note the increased autofluorescence of the nipple (N) in the 700 nm channel. Data are representative of n=3 animals.

### DETAILED DESCRIPTION

The studies described herein demonstrate that platelets loaded with invisible light fluorophores (ILFs), e.g., near-infrared fluorophores (NIRFs), are a sensitive reagent for detecting thrombi in the operative setting. Molecular probes designed to detect thrombi in vivo using IR fluorescence have previously been engineered with a single fluorophore molecule per probe (Jaffer et al., Arterioscler. Thromb. Vasc. Biol., 2002; 22(11):1929-1935; Jaffer et al., Circulation, 2004; 110(2):170-176; Tung et al., Chembiochem., 2002; 3(2-3):207-211). The present invention uses platelets to concentrate ILFs at sites of thrombus formation. For example, IR-786, an exemplary ILF described in these studies, can be concentrated to approximately 3×10⁶ molecules of fluorophore per platelet, corresponding to an intracellular concentration of approximately 700 μM. This enormous concentration of probe compensates for the fact that the fluorescence yield from IR-786 incorporated into platelets is significantly lower than that of free IR-786 in methanol or aqueous buffer (Table 1) due to quenching and internal absorption (Nakayama et al., Mol. Imaging., 2003; 2(1):37-49). In addition to concentrating the ILF, the platelet is uniquely adapted as a probe for detecting blood clots because of its ability to adhere to sites of vascular injury and incorporate into thrombi. This biological signal amplification enables thrombi to be imaged while circulating ILF-loaded platelets remain undetectable. Another advantage of using the platelet as a probe to detect clots formed during surgery is that platelets are the dominant constituent of thrombi. Since platelets concentrate the ILFs and are the major cellular constituent of arterial thrombi, loading of only approximately 2% of platelets (FIGS. 2-6) provides a robust signal that can be visualized intraoperatively.

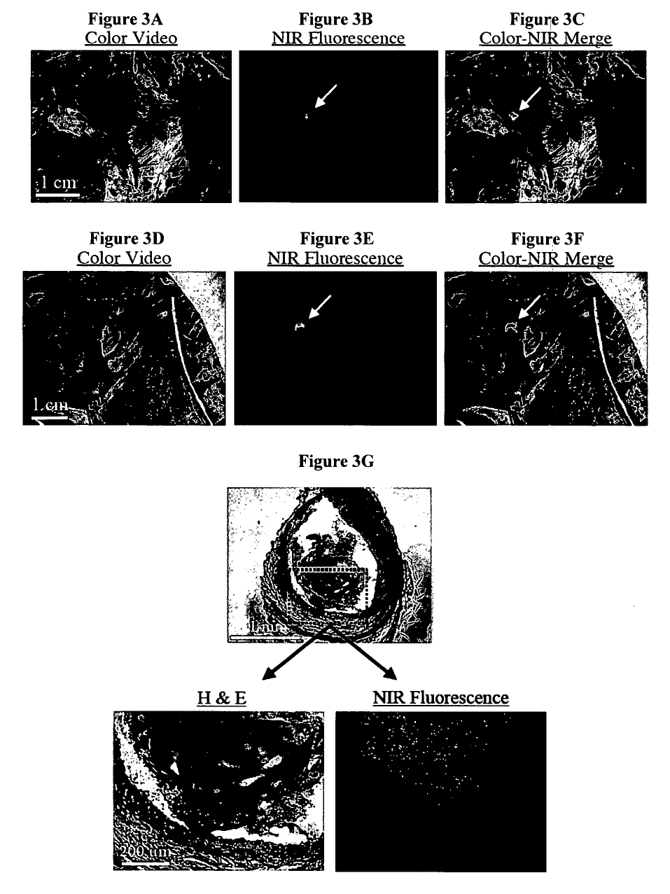

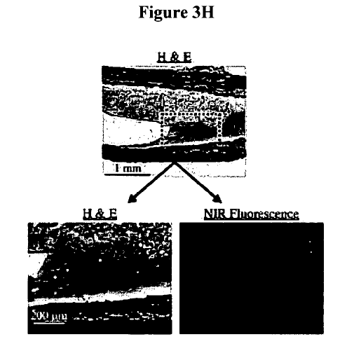

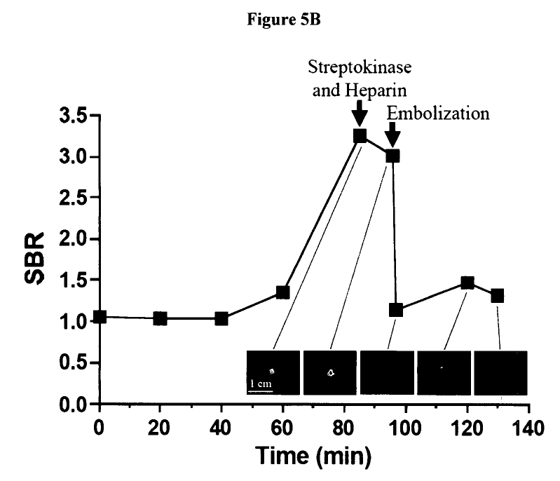

ILF-loaded platelets are a versatile contrast agent. They are capable of detecting thrombi in both arteries and veins (FIGS. 3A-G and 4A-B). They can detect thrombus formation in large, thick walled vessels as well as in small vessels. Since the ILF does not appear to transfer to underlying vascular structures during thrombus formation, ILF-loaded platelets can be used to measure the kinetics of thrombolysis as well as thrombus formation (FIGS. 5A-B).

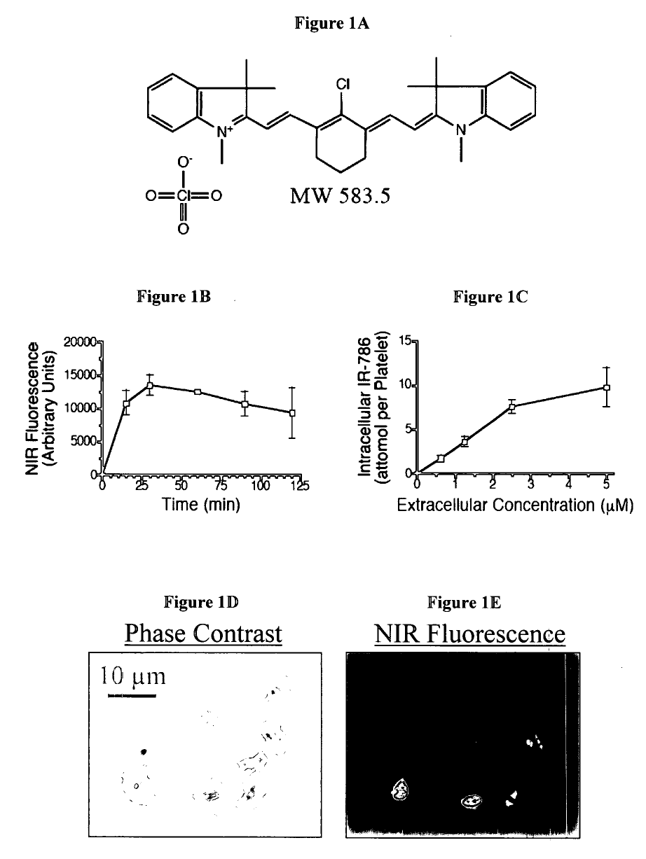

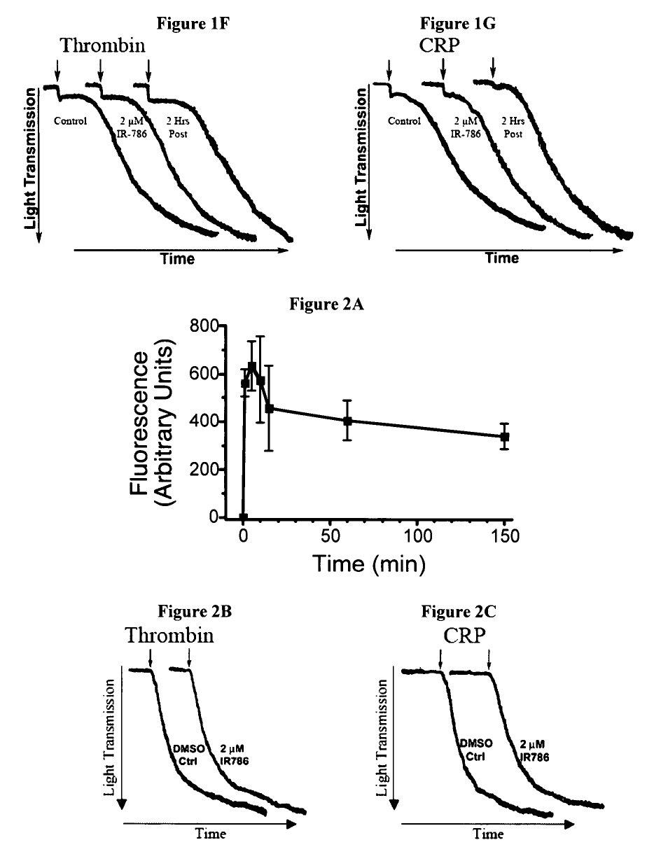

An additional attribute of ILF-loaded platelets is they appear to have a relatively long half-life following infusion into the circulation. More than 50% of fluorescence associated with ILF-loaded platelets could be recovered 150 minutes following infusion into a pig. Much of the decrease in fluorescence appears to be loss of intrinsic fluorescence rather than platelet clearance. This supposition is based partially on the observation that in vitro studies demonstrate a loss of fluorescence of IR-786 within platelets over time (FIG. 1B). Furthermore, manual counting of IR-786-loaded platelets at 15 minutes and 150 minutes following infusion showed no significant difference in the number of loaded platelets. Therefore, IR-786-loaded platelets can detect thrombi for at least 2.5 hours following infusion. In addition, platelets within platelet-rich plasma (PRP), which is equivalent to the single donor platelet product widely available in blood banks, can be conveniently loaded following a 30 minute incubation with an ILF. When infused into pigs, ILF-loaded PRP can also detect thrombi in arteries and veins for up to 2.5 hours (data not shown). Taken together, these observations indicate that IR-786-loaded platelets are a practical reagent for intraoperative thrombus detection.

The pre-clinical studies described herein support the use of ILF-loaded platelets during surgery to detect intravascular thrombi. The imaging technique provides precise localization of a clot within the surgical field. Sensitivity of imaging using ILF-loaded platelets is already significantly greater than that of earlier fluorescent probes designed to detect thrombi. It should also be possible to improve sensitivity using alternative fluorophores (i.e., fluorophores with the properties of platelet accumulation, not altering platelet function, and remaining fluorescent in the invisible light region of the spectrum) or by increasing excitation fluence rates of IR or NIR irradiation.

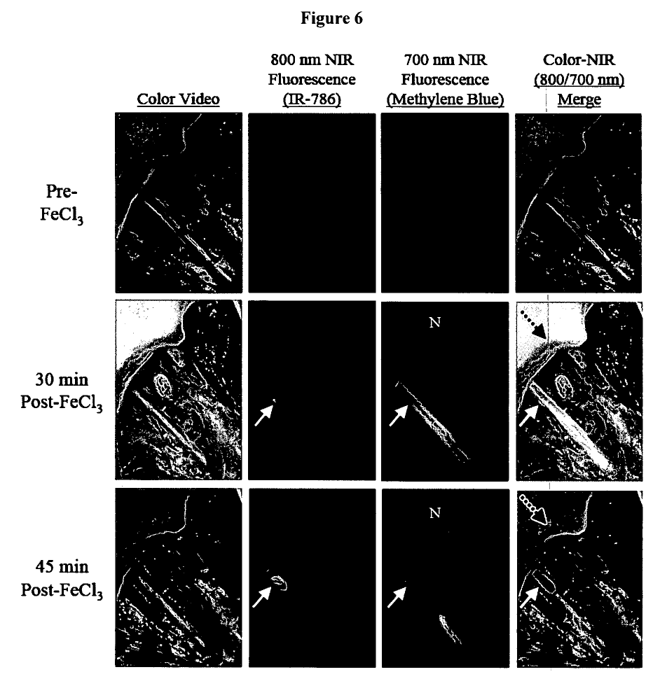

Several features distinguish imaging using ILF-loaded platelets from NIR fluorescence angiography with indocyanine green (ICG) or methylene blue (MB). ICG and MB are cleared rapidly and provide only a negative image of thrombi. Although effective for assessing vessel patency, these dyes are insensitive for detecting thrombi per se and do not accurately localize them (FIG. 6). Detection of thrombus with ILF-loaded platelets provides precise localization of thrombi and can determine whether a thrombus is stable or rapidly evolving. Since ILF-loaded platelets are not cleared rapidly, a single infusion during surgery will be sufficient to detect thrombi throughout the duration of the procedure. Real-time visualization of thrombus during surgery could provide surgeons with valuable information regarding graft patency, anastomosis quality, and detection of thrombosed vessels during invasive vascular procedures, and detection of small sub-occlusive thrombi might be useful in predicting post-operative graft occlusion.

Invisible Light Fluorophores (ILFs)

ILFs fluoresce in the invisible light (IL) region of the spectrum (over about 680 nm, e.g., up to as high as 100,000 nm or higher), such as near infrared (NIR, 680 nm to 1000 nm) to mid infrared (1000 nm to 20,000 nm) to far infrared (20,000 nm to 100,000 nm). Any ILF that (i) can accumulate in platelets at high enough concentrations to give sufficient IL fluorescence, (ii) does not interfere with platelet function when accumulated at concentrations high enough to give sufficient fluorescence, and (iii) retains IL fluorescence once inside the cell and does not interfere with visible light imaging, can be used in the methods described herein. In general, near-infrared fluorophores (NIRFs) are useful.

A number of dyes that can serve as suitable ILFs are known in the art. The suitability of a dye can be readily assayed by incubating platelets with a candidate dye for 30 minutes, and detecting the presence of fluorescence associated with the platelets using methods known in the art, e.g., as described herein, and determining whether the dye interferes with platelet function (e.g., causes or inhibits activation, or interferes with aggregation), using an aggregation assay as described herein.

In some embodiments, the ILF is a heptamethine NIR fluorophore of the indocyanine class. A useful dye is IR-786 (Sigma-Aldrich, Inc.), a commercially available non-sulphonated near-infrared heptamethine indocyanine fluorophore.

In some embodiments, e.g., where the ILF is IR-786, an ILF-loaded platelet includes, for example, at least 1 attomol of ILF per platelet, e.g., at least 2, 3, 4, 5, 6, or more attomoles per platelet, or at least 1×10² ILF molecules per platelet, e.g., at least 1×10³, 1×10⁴, 1×10⁵, 1×10⁶ or 3×10⁶, or more ILF molecules per platelet, and/or has an intracellular concentration of ILF of at least about 1 μM, 10 μM, 100 μM, 350 μM, e.g., about 500 μM, 600 μM, 700 μM, or more.

In some embodiments, an ILF-loaded platelet does not have substantial extracellular labeling with an ILF, but rather accumulates the ILF intracellularly, e.g., in the cytosol, in one or more intracellular organelles, e.g., the endoplasmic reticulum or mitochondria, or in a membrane, e.g., an intracellular membrane or in the plasma membrane. In some embodiments, it is desirable to use a lipophilic, cationic ILF that will be concentrated inside the cell. ILFs suitable for use in these embodiments, wherein the ILF is concentrated inside the cell, include IR-786 and hydrophobic analogs of indocyanine green, IRDye78, IRDye80, IRDye38, IRDye40, IRDye41, IRDye700, IRDye800, Cy5.5, Cy7, and quantum dots, e.g., analogs that lack sulphonation. Hydrophobic analogs will generally be soluble to concentrations ≧10 μM in an organic solvent, such as methanol, but not in an aqueous solvent, such as water or a water-based buffer. Dyes that in their commonly used form do not partition and concentrate in platelets may be modified, e.g., to increase their hydrophobicity, e.g., by decreasing their charge, or adding aliphatic or aromatic groups. See, e.g., Nakayama et al., Molecular Imaging 2(1):37-49 (2003).

In other embodiments, the methods include the use of ILFs conjugated to a cytosol-accumulating moiety, e.g., poly-arginine or the HIV TAT peptide, which direct the ILF to intracellular spaces.

In some embodiments, an ILF-loaded platelet includes an accumulation of ILF on the surface of the cell, e.g., an ILF that is conjugated to the surface of the platelet. For example, an ILF that includes an N-hydroxysuccinimide (NHS) ester group, e.g., IRdye™800CW-NHS, IRdye™78-NHS, Cy5-NHS, Cy5.5-NHS, Cy7-NHS, or Cy7.5-NHS, can be conjugated to amine-containing proteins on the surface of the platelet.

In some embodiments, the methods include the use of a platelet-targeted ILF, e.g., an ILF that is targeted to a platelet by an antigen-binding region of an antibody to a cell-surface protein of the platelet, e.g., CD41, or CD42b. For the purposes of the methods described herein, it is desirable to avoid non-specific binding to Fc receptors present on platelets and other cells. Therefore, the platelet-targeted ILFs will include, e.g., an ILF conjugated to an Fab′₂ portion of an antibody, but will not include an intact IgG.

In general, the ILFs used in the methods and compositions described herein are not calcium-sensitive dyes.

Platelet Preparations

In general, human platelets prepared from normal volunteers can be used in the methods described herein, e.g., immune-matched platelets. In some embodiments, the platelets are obtained from the subject to whom they will be administered, e.g., autologous platelets. Platelets can be washed with physiologic buffers prior to loading with the ILF. Alternatively, platelets within platelet-rich plasma can be loaded with the ILF and used for intraoperative detection of thrombus. ILF-loaded washed platelets may be lyophilized, fixed using fixatives such as paraformaldehyde, or frozen for storage prior to reconstitution and use during surgery. Platelets can be derived from either an autologous or allogeneic source. Single donor or pooled platelet bags from blood banks or commercial sources can be used. Fresh or outdated platelets from blood banks can be used.

An “ILF-loaded platelet” is a platelet that has taken up a sufficient amount of an ILF, i.e., that is coated with or contains an intracellular store of ILFs (e.g., in the mitochondria or endoplasmic reticulum), to be detectable, e.g., when aggregated in a thrombus, adhered to a vessel, or sequestered in an organ, when using an in vivo imaging method described herein. A sufficient amount of an ILF is an amount that is visible by means of a detector above tissue autofluorescence in the emission range of the ILF. A sufficient amount can be determined using methods known in the art, see, e.g., De Grand et al., J. Biomed. Opt. 11(1):014007 (2006), which describes methods for detecting and quantifying autofluorescence of tissues. In some embodiments, a sufficient amount of an ILF has the equivalent fluorescence to 100 nM IRdye™800CW (LI-COR, Lincoln, Nebr.) in a single cell, determined using the system described in De Grand et al., 2006, supra, and Nakayama et al., et al., Molecular Imaging 2(1):37-49 (2003). In some embodiments, a useful concentration is equiv to the fluorescence of at least about 1 μM IRdye™800CW, or at least about 1 μM IRdye™800CW.

Imaging Methods

The methods described herein can be practiced with any intraoperative imaging system that can detect near-infrared fluorescence in vivo, e.g., the systems described in De Grand and Frangioni, Technol. Cancer Res. Treat. 2(6):553-562 (2003); U.S. Pat. App. Pub. No. 2006/0108509 to Frangioni et al.; U.S. Pat. App. Pub. No. 2005/0285038 to Frangioni; U.S. Pat. App. Pub. No. 2005/0020923 to Frangioni et al.; and U.S. Pat. App. Pub. No. 2005/0182321 to Frangioni, all of which are incorporated herein by reference.

The methods described herein can be used as part of an imaging system, e.g., a planar or tomographic imaging system, for high sensitivity detection of fluorescent events, thus, the methods are ideal for intraoperative imaging. Moreover, the methods described herein can be used to provide color, fluorescence and merged image simultaneously in real time, which allows surgeons to keep track of the fluorescence over the surgical field in real time as surgical procedures are ongoing. Depending on the strength of the fluorescence, and the location and size of the structure desired to be imaged, fluorescence that is up to several millimeters from the surface can be detected with planar reflectance imaging. Deeper tissues can be imaged using frequency-domain photon migration or time-domain techniques, which will likely extend depth detection to the 4- to 10-cm range (reviewed in Sevick-Muraca et al., Curr. Opin. Chem. Biol. 6:642-650 (2002) and Ntziachristos et al., Eur. Radiol. 13:195-208 (2003).

Exemplary Indications

In general, the methods described herein can be used to visualize thrombi, e.g., thrombi formation, in vivo. The methods described herein provide information beyond that of previous methods, in that the previous methods generally only provided a static picture of a thrombus, indicating the presence or absence of a thrombus. The present methods provide additional, functional information, because the ILF-loaded platelets described herein only adhere to actively growing thrombi. The accumulation of ILF-loaded platelets allows for the production of a detectable IL fluorescent signal, which indicates the presence of a thrombus.

Thus, the methods described herein can be used for detecting a thrombus in vivo, e.g., an intraoperative thrombus that occurs during a surgical procedure. The methods described herein can also be used for detecting vulnerable plaques in vivo, e.g., in an artery, e.g., in subjects who are suspected of having atherosclerosis, e.g., subjects who have chest pain, and possibly a history of heart disease. These methods can include the use of an angioscope that is equipped to detect both visual light and fluorescence. The normal coronary luminal wall is angioscopically smooth and white, and plaques are defined as vessel areas of white, brown, or yellow color with an irregular or smooth surface. See, e.g., Ueda et al., Vascular Disease Prevention, 1:53-57 (2004). Vulnerable plaques are those that are actively growing and therefore may get to the point where they rupture, e.g., become thrombotic. The ILF-loaded platelets described herein can be used to detect these growing plaques, because platelets will generally only adhere to growing, vulnerable plaques. Therefore, the presence of invisible light emission from the ILF-loaded platelets indicates the presence of a vulnerable plaque.

While it is expected that accumulation of ILF-loaded platelets on a thrombus will happen quickly, it is possible that accumulation on a plaque may take longer. Thus, in some embodiments, methods for detecting vulnerable plaques may include administering ILF-loaded platelets and allowing time for the ILF-loaded platelets to accumulate on a plaque before imaging with an angioscope.

The methods described herein can also be used to detect retained or accessory spleen in vivo. An accessory spleen is a common congenital condition that causes small globular masses of splenic tissue, generally found in the area of the spleen, or in one of the peritoneal folds. A retained spleen is a mass of splenic tissue left in the body after removal of the spleen, e.g., after an injury to the spleen. The ILF-loaded platelets will accumulate in the retained or accessory spleen, therefore, the presence of invisible light emission from the ILF-loaded platelets can indicate the presence of a retained or accessory spleen.

Finally, because the ILF-loaded platelets aggregate at the site of actively forming thrombi, the methods described herein can be used to determine whether intraoperative bleeding is due to a clotting disorder or is normal surgical bleeding. In general, bleeding that is due to a clotting disorder will not be associated with clot formation. If there is no clot formation, there will be no accumulation of ILF-loaded platelets and no detectable IL fluorescence. Normal surgical bleeding, on the other hand, will be associated with clot formation. Accumulation of ILF-loaded platelets at the site of active clot formation will lead to detectable IL fluorescence.

Currently, a primary test for diagnosis of a clotting disorder is the Bleeding Time test, in which a small cut is made on the person's forearm, and the examiner measures the amount of time that elapses before bleeding stops, a relatively subjective measure. Therefore, detecting the presence of a clotting disorder can be enhanced using the present methods, which can be used to detect and accurately monitor clot formation in real time.

### EXAMPLES

The invention is further described in the following examples, which do not limit the scope of the invention described in the claims.

### Example 1

### Incorporation of IR-786 into Human Platelets

To develop a contrast agent capable of detecting thrombi during surgery, platelets were loaded with IR-786, a highly hydrophobic non-sulfonated heptamethine dye that emits NIR light (structure shown in FIG. 1A).

Washed human platelets were prepared from fresh blood obtained from aspirin-free donors by differential centrifugation as described previously (Sim et al., Blood, 2004; 103: 2127-2134; Jaffer et al., Arterioscler. Thromb. Vasc. Biol., 2002; 22(11):1929-1935). Washed pig platelets were isolated from fresh blood obtained from anaesthetized Yorkshire pigs. Platelet-rich plasma (PRP) was prepared by centrifugation at 200×g for 20 minutes. Platelets were then isolated from PRP by centrifugation at 1,400×g for 10 minutes in the presence of 50 ng/mL PGE₁ and 10% (v/v) acid citrate/dextrose, pH 4.6, and resuspended at a concentration of 4×10⁸ cells/mL in Tyrodes-HEPES buffer (134 mM NaCl; 0.34 mM Na₂HPO4; 2.9 mM KCl; 12 mM NaHCO₃; 20 mM HEPES; 5 mM glucose and 1 mM MgCl₂, pH 7.3). The perchlorate salt of IR-786 (CAS #102185-03-5) was purchased from Sigma-Aldrich (St. Louis, Mo.).

After being added to an extracellular medium, IR-786 incorporates rapidly into intracellular membranes, with a concentration-dependent partitioning into mitochondria and/or endoplasmic reticulum (Nakayama et al., Mol. Imaging., 2003; 2(1):37-49). Total fluorescence yield increases markedly in such lipid rich environments, but quenching can, and does, occur at high intracellular concentrations (Nakayama et al., Mol. Imaging., 2003; 2(1):37-49).

To quantify uptake of IR-786 into platelets, platelet counts were measured using the HEMAVET Multispecies Hematology Analyzer (Drew Scientific, Oxford, Conn.). For platelet loading experiments, 1 mL samples of washed platelets (approximately 4×10⁸ total) were incubated for 0, 15, 30, 60, 90, or 120 minutes, at room temperature, with gentle rocking, in Tyrodes-HEPES supplemented with 5, 2.5, 1.25, 0.625 or 0 μM IR-786. For measurements, platelets were pelleted for 5 minutes at 2,000×g in the presence of PGE₁. Pellets were lysed with 500 μL absolute methanol by repeated pipetting and sonication for 1 minute at a 50% duty cycle. Sample fluorescence was measured using the imaging system described below by comparison to IR-786 calibration standards in methanol (pellets) or Tyrodes-HEPES (supernatant).

Absorbance and fluorescence measurements were performed in 1 cm quartz cuvettes as described previously (Ohnishi et al., Mol. Imaging., 2005; 4(3):172-181). For measurement of relative fluorescence yield, IR-786 samples in Tyrodes-HEPES, methanol, or concentrated in washed platelets were matched for absorbance (0.1 A units) and area under the fluorescence emission curve calculated after excitation with a 5 mW 655 nm laser diode.

Time course studies demonstrated maximal incorporation of IR-786 into platelets following 30 minutes of incubation (FIG. 1B). Loading of IR-786 beyond this time point resulted in lower total accumulation, possibly due to osmotic effects and/or dye aggregation. Spectral analysis demonstrated that platelet incorporation of IR-786 resulted in a characteristic red shift. The excitation and emission maxima of IR-786 in aqueous buffer were 769.3 nm and 788.8, respectively (Table 1). In contrast, its excitation and emission maxima following incorporation into platelets were 788 nm and 804 nm, even redder than emission found in neat methanol (Table 1).

TABLE 1
Spectral characteristics and relative fluorescence yield of IR-786 as
a function of local chemical environment

| | | Peak | Relative |
|---|---|---|---|
| Buffer or Cell | Peak Absorbance | Emission | Fluorescence Yield |
| Tyrode's-HEPES | 769 nm | 789 nm | 1.4 |
| Methanol | 775 nm | 797 nm | 9.5 |
| Platelets | 788 nm | 804 nm | 1.0 |

To determine the optimal fluorescence yield following platelet loading, platelets were exposed to increasing concentrations of IR-786. Loading of platelets occurred in a linear manner until 2.5 μM (FIG. 1C). For all concentrations tested, only trace fluorescence remained in the supernatant. At concentrations >2.5 μM, the linearity of dose-dependency was lost indicating self-quenching and/or dye aggregation. All subsequent experiments were therefore performed using 2 μM IR-786. At this concentration, platelets incorporated approximately 6 attomoles (roughly 3,600,000 molecules) of IR-786 per platelet.

The subcellular location of IR-786 was characterized in loaded platelets. NIR fluorescence microscopy was performed on a four filter set Nikon Eclipse TE-300 epifluorescence microscope as previously described (Nakayama et al., Mol. Imaging., 2003; 2(1):37-49).

Although there was a small degree of homogenous staining of lamellipodia and pseudopodia consistent with plasma membrane staining, the majority of fluorescence was punctate and localized in the central granulomere (FIGS. 1D-E).

This pattern of fluorescence suggests incorporation of dye into intracellular structures and is consistent with staining patterns observed in other cell types (Nakayama et al., Mol. Imaging., 2003; 2(1):37-49).

### Example 2

### Human Platelet Aggregation Studies

To ensure that platelets loaded with 2 μM IR-786 retained function, the ability of IR-786-loaded platelets to aggregate in response to agonists was assessed. Loaded and non-loaded human platelets, resuspended at a density of 4×10⁸ cells/mL in modified Tyrodes-HEPES buffer, were stimulated with agonists in siliconized glass tubes in an optical aggregometer (Chronolog, Havertown, Pa.). Assays were performed at 37° C. and with constant stirring. Aggregation was monitored by measurement of optical density of the platelet suspension (Croce et al., J. Biol. Chem., 1999; 274(51):36321-36327)

IR-786-loaded platelets demonstrated normal aggregation in response to either thrombin or collagen related protein (CRP; FIG. 1F-G). Evaluation of resting platelets loaded with 2 μM IR-786 showed no significant P-selectin surface expression demonstrating that incubation with IR-786 does not activate platelets.

These data indicate that platelets remain functionally intact following loading.

### Example 3

### Clearance of IR-786-Loaded Platelets In Vivo

A porcine model was used to test the clearance of platelets loaded with IR-786.

Yorkshire pigs (E. M. Parsons and Sons, Hadley, Mass.) weighing 35 kg were induced for anesthesia with 4.4 mg/kg IM tiletamine/zolazopam (Telazol, Fort Dodge Labs, Fort Dodge, Iowa). Once sedated, animals were intubated using a 7-mm cuffed endotracheal tube, and anesthesia maintained using oxygen and isoflurane 0.5 to 5% to effect. Animals were prepped and draped in the usual sterile fashion, and the vessels indicated exposed using standard surgical techniques.

Washed platelets were prepared from 200 ml whole blood obtained from an anesthetized 35 kg Yorkshire pig. Pig platelets were loaded with 2 μM IR-786 for 30 minutes. IR-786-loaded platelets (3.6×10¹⁰) were then infused through a cannula in the internal jugular vein of the pig and the line was extensively flushed with saline. Blood samples were obtained at 1, 5, 10, 15, 60, and 150 minutes following infusion, and platelet-rich plasma was analyzed.

Evaluation of platelet-associated fluorescence, performed as described above, demonstrated a rapid increase in fluorescence following infusion of the IR-786-loaded platelets (FIG. 2A). Following this increase, there was a period of sharp decline in fluorescence until approximately 20 minutes. A slow decline in fluorescence then followed. Clearance of IR-786-loaded platelets was also analyzed by manual counting of loaded and non-loaded platelets in PRP. Microscopic analysis showed that 2.0±0.4% of platelets were loaded at 15 minutes following infusion and that 2.6±0.6% (p=0.4) were loaded at 150 minutes following infusion. Based on these data, a majority of IR-786-loaded platelets remain circulating 150 minutes following infusion into pigs. The decline in fluorescence observed in FIG. 2A may not be primarily due to platelet clearance; free IR-786 is likely to be rapidly cleared by the liver following infusion (Nakayama et al., Mol. Imaging., 2003; 2(1):37-49) and the fluorescent signal in the platelets slowly declines over time as demonstrated in FIG. 1C.

Overall, these data indicate that the majority of IR-786-loaded pig platelets remain in the circulation.

The ability of pig platelets to aggregate following incubation with IR-786 was also tested, using aggregometry as described above. Washed pig platelets incubated for 30 minutes with 2 μM IR-786 aggregated normally in response to thrombin or CRP (FIGS. 2B-C). In addition, evaluation of aggregometry of pig platelets obtained 2 hours after infusion of IR-786-loaded platelets demonstrated that loading with 2 μM IR-786 did not affect platelet aggregation. Furthermore, NIR microscopy of platelets following aggregation studies demonstrated that IR-786-loaded platelets incorporate into aggregates. Evaluation of erythrocyte and platelet counts following infusion of IR-786-loaded platelets demonstrated that infusion of this contrast media had no significant effects on circulating numbers of these blood cells.

These results demonstrate that 2 μM IR-786 does not inhibit aggregation of pig platelets.

### Example 4

### IR-786-Loaded Platelets Detect Thrombus Formation Intraoperatively

Next, it was determined whether the methods could be used to detect thrombi in live pigs, and in real time, using IR-786-loaded platelets. Thrombus formation following oxidant injury induced by exposure of vessels to filter paper saturated with FeCl₃ is a widely used and reliable method for induction of thrombus formation in vivo (Frenette et al., Proc. Natl. Acad. Sci. U.S.A., 1995; 92(16):7450-7454; Dogne et al., Thromb. Res., 2005; 116(5):431-442; Kurz et al., Thromb. Res., 1990; 60(4):269-280). It was hypothesized that the high fluorescence yield of IR-786 in platelets and the enhanced tissue penetration of NIR light would enable us to visualize thrombi even in large, thick-walled vessels.

Platelets (3.6×10¹⁰ total) in Tyrode's-HEPES were loaded with 2 μM IR-786 for 30 min at room temperature and infused intravenously prior to induction of thrombi using either FeCl₃, embolic coil, intravascular stent, or cutaneous incision. For induction of thrombus formation using FeCl₃, a 0.5×1 cm swatch of grade 413 Whatman filter paper was saturated with a 50% solution of ferric chloride (Sigma-Aldrich) and applied beneath the vessel so as not to impede visualization of IR-786-loaded platelet accumulation. For induction of thrombi with either embolic coils or stents, a 5 Fr PINNACLE™ sheath introducer (Terumo Medical, Elkton, Md.) was inserted into the vessel and used to deploy devices. Thrombus formation was then imaged continuously until fluorescence signal stabilized.

The imaging system has been described in detail previously (De Grand and Frangioni, Technol. Cancer Res. Treat., 2003; 2(6):553-562), with the following modifications. Three wavelength-isolated excitation sources were utilized, one generating 400 to 680 nm “white” light (0.5 mW/cm²), a second generating 680-700 nm low-NIR fluorescence excitation light (1 mW/cm²) utilizing model #L-660-66-60-550, high power light emitting diodes (Marubeni Epitex, New York, N.Y.) and custom excitation filters, and a third generating 725-775 nm NIR fluorescence-excitation light (5 mW/cm²), all over a 15-cm diameter field of view (FoV). Photon collection was achieved with custom-designed optics that maintain the separation of the white light and NIR fluorescence emission (i.e., 700-725 nm or >795 nm) channels. After computer-controlled camera acquisition via custom LabVIEW (National Instruments, Austin, Tex.) software, anatomic (white light) and functional (NIR fluorescence light) images could be displayed separately and merged. To create a single, merged image that displayed both anatomy (color video) and function (NIR fluorescence), the NIR fluorescence image was pseudo-colored (e.g., in lime green) and overlaid with 100% transparency on top of the color video image of the same surgical field. All images were refreshed up to 15 times per second. The entire apparatus was suspended on an articulated arm over the surgical field, thus permitting non-invasive and non-intrusive imaging.

Vessel patency was assessed by intravenous injection of 1 ml of 1% (10 mg total) methylene blue (Mayne Pharma, Paramus, N.J.) with continuous imaging of NIR fluorescence (700-725 nm) emission.

For quantification of in vivo thrombi, NIR fluorescence excitation fluence rate and FoV were held constant for all quantitative comparisons. Regions of interest (ROI) of a defined shape and pixel number could be moved anywhere within the FoV to quantify NIR fluorescence emission signal intensity from the 12-bit camera. Signal to background ratio (SBR) was assessed by quantifying fluorescence signal from an ROI encompassing the thrombus compared with an intravascular ROI of the same size proximal to the thrombus.

FeCl₃-induced injuries to the femoral arteries were studied first. Imaging demonstrated the accumulation of platelets at the site of injury as represented by increased fluorescence signal (FIG. 3A-3C). Development of platelet-rich thrombi could also be visualized following FeCl₃-induced injury of the femoral vein (FIG. 3D-3F). Quantification of images demonstrated that platelet accumulation began 25-35 minutes following application of FeCl₃ (see below). This delay may represent the time required for diffusion of the FeCl₃ through the vessel wall and/or the time required for oxidative denudation of the endothelium. Thrombus formation began following this delay and continued to increase over the 150 min experiment (see, for example, FIG. 5B). The maximum signal to background ratio varied between thrombi from approximately 2 to 12 depending on fluence rate and FoV. These studies demonstrated that IR-786-loaded platelets accumulate at sites of thrombus formation, thereby providing precise localization of thrombi within large, thick-walled vasculature.

Hematoxylin and eosin staining of these vessels demonstrated large thrombi oriented towards the portion of the vessel exposed to FeCl₃ (FIGS. 3G-3H). NIR microscopy showed that IR-786-loaded platelets were diffusely incorporated throughout the body of the thrombus (FIGS. 3G-H). There was no evidence for incorporation of IR-786 into the underlying vasculature, indicating that IR-786 remained platelet-associated.

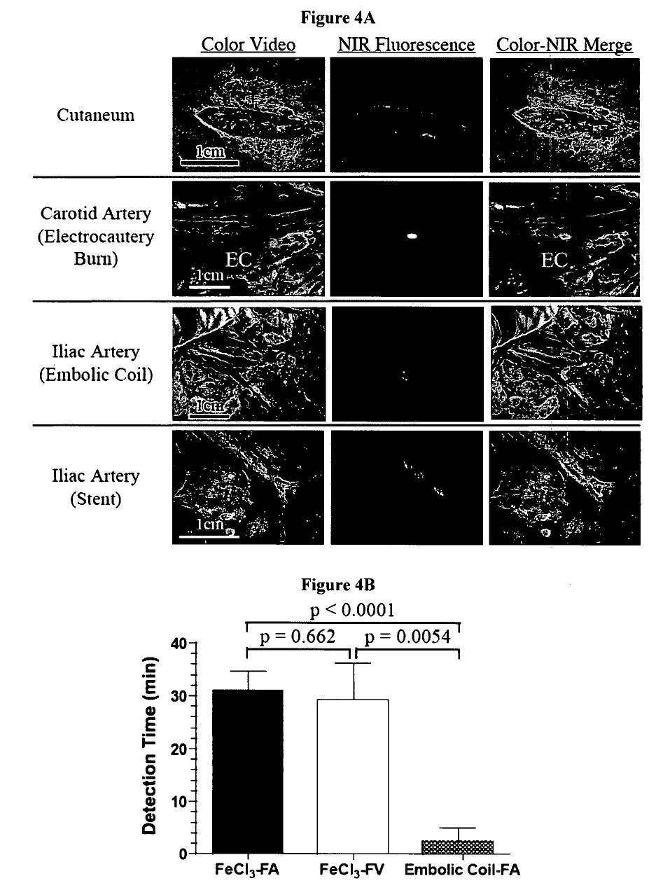

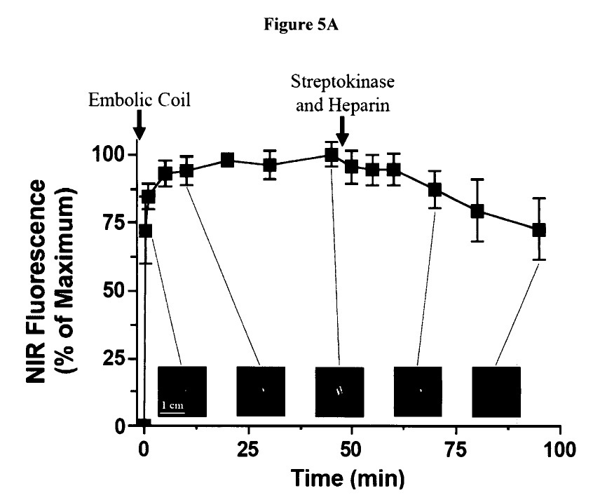

Although the FeCl₃-induced injury is widely used to model thrombus formation in vivo, further experiments were performed to determine whether IR-786-loaded platelets could detect thrombi formed under circumstances encountered during surgical or vascular procedures. Following cutaneous incision and wound irrigation, a rim of thrombus formation could be visualized at the edge of wounds (FIG. 4A, top row). Occasionally, a thrombus would form at the site of electrocautery, as is shown in FIG. 4A, second row, for the carotid artery. Thrombus formation reproducibly occurred following insertion of intravascular devices into major vessels. Placement of an embolic coil into the ileac artery in an unheparinized animal resulted in rapid thrombus formation (FIG. 4A, third row). Similarly, a thrombus developed in the ileac artery following placement of a stent in an unheparinized animal (FIG. 4A, bottom row).

The onset of thrombus formation following surgical manipulation of vessels or placement of intravascular devices was significantly more rapid than that following FeCl₃ exposure (FIG. 4B). Time to first detected thrombus in the femoral artery and femoral vein treated with FeCl₃ was 31.0±3.58 min and 29.17±7.12 min, respectively. Time to first detected thrombus for an embolic coil placed in the femoral artery was 2.50±2.50 min. The average maximal SBR following FeCl₃ exposure was 4.4±1.7 compared with an average maximal SBR of 3.3±1.9 following embolic coil placement.

These examples demonstrate that IR-786-loaded platelets constitute a versatile and quantitative contrast medium for detecting thrombi formed following a variety of vascular manipulations.

### Example 5

### Monitoring Thrombolysis with IR-786-Loaded Platelets

IR-786-loaded platelets were used to monitor the dynamics of thrombus growth and dissolution intraoperatively. Thrombolytics streptokinase and heparin were used. Streptokinase was obtained from Sigma-Aldrich and heparin was obtained from American Pharmaceutical Partners (Schaumburg, Ill.).

As shown in FIG. 5A, placement of an embolic coil in the femoral artery resulted in rapid formation of an intravascular thrombus, the extent of which could be quantified using NIR fluorescence. By 40 minutes, the thrombus had stabilized in size, at which point streptokinase and heparin were infused, and dissolution of the thrombus was monitored in real-time. A second pattern of thrombus behavior after streptokinase and heparin infusion is shown in FIG. 5B for a femoral artery treated with FeCl₃. In this case, thrombolytics triggered embolization of the thrombus, which then reformed slowly in the vessel.

These data demonstrate that IR-786-loaded platelets can be used to monitor the efficacy of thrombolytic therapy in vivo and in real-time.

### Example 6

### Assessment of Vascular Patency During Thrombus Formation

Detection of an intravascular thrombus, even a large one, does not necessarily result in cessation of blood flow. One of the many advantages of NIR light is that the “NIR window (700-900 nm)” (Chance, Ann. N.Y. Acad. Sci., 1998; 838:29-45) is 200 nm wide. This permits more than one NIR fluorophore to be used simultaneously. In recent work (Tanaka et al., manuscript in preparation), the NIR fluorescent properties of methylene blue (MB), an agent already FDA-approved as a blue dye for surgery, were characterized. Since MB fluorescence peaks at approximately 700 nm, its fluorescence is well separated from that of IR-786.

As shown in FIG. 6, a thrombus is seen growing in the vessel until vascular occlusion occurs, at which point the vessel is supplied only by back-fill through a small collateral.

MB can thus be used to assess vessel patency simultaneously with IR-786 loaded platelets to assess thrombus size and location.

### REFERENCES

1. Falati et al., Nat. Med., 2002; 8(10):1175-1181
2. Frenette et al., Proc. Natl. Acad. Sci. U.S.A., 1995; 92(16):7450-7454
3. Sim et al., Blood, 2004; 103(6):2127-2134
4. Chou et al., Blood, 2004; 104(10):3190-3197
5. Day et al., Blood, 2005; 105(1):192-198
6. Seiffge et al., Thromb. Res., 1986; 42(3):331-341
7. Falk et al., J. Card. Surg., 1995; 10(2):147-160
8. Hol et al., Ann. Thorac. Surg., 2004; 78(2):502-505; discussion 505
9. D'Ancona et al., Heart Surg. Forum, 2000; 3(2):99-102
10. Gundry et al., J. Thorac. Cardiovasc. Surg., 1998; 115(6):1273-1277; discussion 1277-1278
11. Acland, Surgery, 1973; 73(5):766-771
12. Serletti et al., Plast. Reconstr. Surg., 1998; 102(6):1947-1953
13. Reuthebuch et al., Chest, 2004; 125(2):418-424
14. Frangioni, Curr. Opin. Chem. Biol., 2003; 7(5):626-634
15. Balacuaswami et al., J. Thorac. Cardiovasc. Surg., 2005; 130(2):315-320
16. Rozenvayn and Flaumenhaft, J. Biol. Chem., 2003; 278(10):8126-8134
17. Ohnishi et al., Mol. Imaging., 2005; 4(3):172-181
18. Nakayama et al., Mol. Imaging., 2003; 2(1):37-49
19. Croce et al., J. Biol. Chem., 1999; 274(51):36321-36327
20. De Grand and Frangioni, Technol. Cancer Res. Treat., 2003; 2(6):553-562
21. Dogne et al., Thromb. Res., 2005; 116(5):431-442
22. Kurz et al., Thromb. Res., 1990; 60(4):269-280
23. Chance, Ann. N.Y. Acad. Sci., 1998; 838:29-45
24. Jaffer et al., Arterioscler. Thromb. Vasc. Biol., 2002; 22(11):1929-1935
25. Jaffer et al., Circulation, 2004; 110(2):170-176
26. Tung et al., Chembiochem., 2002; 3(2-3):207-211
27. Baum et al., J. Invest. Surg., 2001; 14(3):153-160
28. Kim et al., Acta. Neurochir. (Wien), 2004; 146(1):45-51; discussion 51

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of detecting an aggregation of platelets within a living subject, the method comprising:
administering to a subject a sufficient amount of a composition comprising invisible light fluorophore (ILF)-loaded platelets; and
detecting invisible light emission from the ILF-loaded platelets,
wherein the presence of invisible light emission from the ILF-loaded platelets indicates the presence of an aggregation of platelets.

2. The method of claim 1, wherein detection of invisible light (IL) emission from the ILF-loaded platelets is during a surgical procedure.

3. The method of claim 1, wherein the aggregation of platelets is associated with a thrombus.

4. The method of claim 1, wherein the aggregation of platelets is associated with a vulnerable plaque.

5. The method of claim 4, wherein the invisible light emission is detected using an angioscope.

6. The method of claim 1, wherein the aggregation of platelets is associated with a presence of retained or accessory spleen.

7. A method of determining whether intraoperative bleeding in a surgical field is due to a clotting disorder or is normal surgical bleeding in vivo, the method comprising:
administering to a subject a sufficient amount of a composition comprising invisible light fluorophore (ILF)-loaded platelets; and
detecting invisible light emission from the ILF-loaded platelets in the surgical field, wherein the presence of invisible light emission from the ILF-loaded platelets in the surgical field indicates that the bleeding is normal surgical bleeding, and the absence of invisible light emission from the ILF-loaded platelets in the surgical field indicates that the bleeding is due to a clotting disorder.

8. A method of evaluating a subject for the presence of a clotting disorder, the method comprising:
administering to a subject a sufficient amount of a composition comprising invisible light fluorophore (ILF)-loaded platelets; and
detecting invisible light emission from the ILF-loaded platelets, wherein the presence of invisible light emission from the ILF-loaded platelets indicates that the subject does not have a clotting disorder, and the absence of invisible light emission from the ILF-loaded platelets indicates that the subject has a clotting disorder.

9. The method of claim 1, wherein the platelets are autologous to the subject.

10. The method of claim 1, wherein the platelets are allogeneic to the subject.

11. The method of claim 1, wherein the platelets are from a blood-bank or other commercial source.

12. The method of claim 1, wherein the ILF is a near-infrared fluorophore.

13. The method of claim 1, wherein the ILF is IR-786.

14. The method of claim 1, wherein detecting invisible light emission from the ILF-loaded platelets comprises illuminating the subject with an excitation wavelength of the ILF; and electronically capturing a IL emission wavelength image of the ILF.

15. The method of claim 7, wherein the platelets are autologous to the subject.

16. The method of claim 7, wherein the platelets are allogeneic to the subject.

17. The method of claim 7, wherein the platelets are from a blood-bank or other commercial source.

18. The method of claim 7, wherein the ILF is a near-infrared fluorophore.

19. The method of claim 7, wherein the ILF is IR-786.

20. The method of claim 7, wherein detecting invisible light emission from the ILF-loaded platelets comprises illuminating the subject with an excitation wavelength of the ILF; and electronically capturing a IL emission wavelength image of the ILF.

21. The method of claim 8, wherein the platelets are autologous to the subject.

22. The method of claim 8, wherein the platelets are allogeneic to the subject.

23. The method of claim 8, wherein the platelets are from a blood-bank or other commercial source.

24. The method of claim 8, wherein the ILF is a near-infrared fluorophore.

25. The method of claim 8, wherein the ILF is IR-786.

26. The method of claim 8, wherein detecting invisible light emission from the ILF-loaded platelets comprises illuminating the subject with an excitation wavelength of the ILF; and electronically capturing a IL emission wavelength image of the ILF.

27. A composition comprising a plurality of invisible light fluorophore (ILF)-loaded platelets.

28. The composition of claim 27, wherein the ILF is concentrated in a membrane; in an organelle; in cytosol, or on the surface of the platelet.

29. The composition of claim 27, wherein the membrane is a plasma membrane or an intracellular membrane

30. The composition of claim 27, wherein the organelle is a mitochondria, endoplasmic reticulum, or a nucleus.

31. The composition of claim 27, wherein the ILF is a near-infrared fluorophore (NIRF).

32. The composition of claim 27, wherein the ILF is IR-786.

33. The composition of claim 27, wherein the platelets are fresh, lyophilized, fixed or frozen.

34. The composition of claim 27, wherein the platelets are in platelet-rich plasma.

35. The composition of claim 27, wherein the platelets are from a blood-bank or other commercial source.

36. A method for preparing invisible light fluorophore (ILF)-loaded platelets, the method comprising incubating a composition comprising platelets with an ILF under conditions and for a length of time sufficient for the platelets to take up the ILF.

37. The method of claim 36, wherein the ILF is a near-infrared fluorophore (NIRF).

38. The method of claim 36, wherein the ILF is IR-786.

39. The method of claim 36, wherein the composition comprises platelet-rich plasma (PRP).

40. A kit for preparing invisible light fluorophore (ILF)-loaded platelets, the kit comprising a container including a sterile composition comprising an ILF and instructions for use in the method of claim 1.

41. A kit for preparing invisible light fluorophore (ILF)-loaded platelets, the kit comprising a container including a sterile composition comprising an ILF and instructions for use in the method of claim 7.
